(19) United States
(12) Patent Application Publication
(10) Pub. No.: US 2022/0249862 A1
(43) Pub. Date: Aug. 11, 2022

(54) VITAMIN D SYNTHESIS AND STERILIZATION CONTROL DEVICE AND VITAMIN D SYNTHESIS AND STERILIZATION CONTROL METHOD OF VITAMIN D SYNTHESIS AND STERILIZATION CONTROL DEVICE

(71) Applicant: CEBIEN CO., LTD, Gyeonggi-do (KR)

(72) Inventors: Bae Dong JHIN, Gyeonggi-do (KR); Byeong Seol GO, Gyeonggi-do (KR)

(21) Appl. No.: 17/374,077

(22) Filed: Jul. 13, 2021

(30) Foreign Application Priority Data
Feb. 8, 2021 (KR) 10-2021-0017409
Feb. 25, 2021 (KR) 10-2021-0025575

(51) Int. Cl. A61N 5/06; A61L 2/00

(52) U.S. Cl. CPC A61N 5/0616; A61N 2005/0661; A61L 2/0088; A61L 2/0047

(57) ABSTRACT

A vitamin D synthesis and sterilization control device according to an embodiment may include a first light source configured to irradiate light for synthesizing vitamin D in the body of a user, a second light source configured to irradiate light for sterilizing a bathroom, a first sterilizing water discharging unit configured to discharge a low-concentration sterilizing water for washing the user, a second sterilizing water discharging unit configured to discharge a high-concentration sterilizing water for cleaning the bathroom, a sensor unit configured to detect whether the user is in the bathroom, and a control unit configured to generate an operation command for controlling at least one of the first light source, the second light source, the first sterilizing water discharging unit and the second sterilizing water discharging unit to operate based on the detection result on whether the user is in the bathroom.

### CROSS-REFERENCE TO RELATED APPLICATION AND CLAIM OF PRIORITY

This application claims priority to Korean Patent Applications Nos. 10-2021-0017409 filed on Feb. 8, 2021 and 10-2021-0025575 filed on Feb. 25, 2021 in the Korean Intellectual Property Office (KIPO), the entire disclosure of which is incorporated by reference herein.

### BACKGROUND

### 1. Technical Field

This disclosure relates to a vitamin D synthesis and sterilization control device and a vitamin D synthesis and sterilization control method of the vitamin D synthesis and sterilization control device, and more specifically, to a vitamin D synthesis and sterilization control device for selectively controlling vitamin D synthesis and/or sterilization based on a detection result on whether a user is in a bathroom, and a vitamin D synthesis and sterilization control method of the vitamin D synthesis and sterilization control device.

### 2. Background Art

Recently, health has emerged as the most important issue in the daily life of modern people.

For example, as the proportion of living indoors is gradually increasing, it is common for modern people who lack sunlight to have insufficient opportunities to synthesize vitamin D.

Accordingly, various products such as light sources for vitamin D synthesis are being released.

However, most of the vitamin D synthesis products distributed on the market so far are used in a state where a user wears the product on the body separately, and for this reason, the user gradually feels cumbersome to use the product and often neglects the use of the product.

In addition, a conventional vitamin D synthesis product is used without considering an actual amount of vitamin D synthesis required by the user, so it is difficult for the user to set the time for using the product.

Meanwhile, recently, a virus with high contagious and lethality, such as COVID-19, has emerged, which not only causes significant loss of life and but also leads to huge economic losses for the country due to weakened consumption life caused by restrictions on movement and psychological atrophy as well as a huge amount of manpower and goods for quarantine.

In order to effectively protect the human body from viruses and various harmful bacteria that spread these diseases, thorough personal hygiene must be practiced, and moreover thorough quarantine of mediators is required to fundamentally solve the spread of viruses or bacteria.

Therefore, in these days when the aforementioned health for the human body is emerging as the most important issue, there is a need to easily perform vitamin D synthesis without user manipulation or additional difficulties and to safely perform virus sterilization.

### SUMMARY

The present disclosure is designed by reflecting the aforementioned needs, and the present disclosure is directed to enabling vitamin D synthesis be easily performed without user manipulation or additional difficulties.

In particular, the present disclosure is directed to providing a device to automatically synthesize vitamin D while a user is washing for virus sterilization in a bathroom environment.

In addition, the present disclosure is directed to handling the device more efficiently by allowing the device to perform functions other than cleaning the user or performing vitamin D synthesis for the user when the user is not in the bathroom.

A vitamin D synthesis and sterilization control device according to an embodiment may include a first light source configured to irradiate light for synthesizing vitamin D in the body of a user; a second light source configured to irradiate light for sterilizing a bathroom; a first sterilizing water discharging unit configured to discharge a low-concentration sterilizing water for washing the user; a second sterilizing water discharging unit configured to discharge a high-concentration sterilizing water for cleaning the bathroom; a sensor unit configured to detect whether the user is in the bathroom; and a control unit configured to generate an operation command for controlling at least one of the first light source, the second light source, the first sterilizing water discharging unit and the second sterilizing water discharging unit to operate based on the detection result on whether the user is in the bathroom.

When the sensor unit detects that the user is in the bathroom, the control unit may generate an operation command for controlling at least one of the first light source and the first sterilizing water discharging unit to operate.

When the sensor unit detects that the user is not in the bathroom, the control unit may generate an operation command for controlling at least one of the second light source and the second sterilizing water discharging unit to operate.

The control unit may generate the operation command when at least one of the first light source, the second light source, the first sterilizing water discharging unit and the second sterilizing water discharging unit is activated.

The vitamin D synthesis and sterilization control device may further include a communication unit configured to receive an activation command for activating at least one of the first light source, the second light source, the first sterilizing water discharging unit and the second sterilizing water discharging unit from a portable terminal, and the control unit may control to perform the activation based on the activation command.

The vitamin D synthesis and sterilization control device may further include a communication unit configured to receive biometric information of the user from a portable terminal, and the control unit may control to change at least one of an amount of the light irradiated by the first light source and an amount of the low-concentration sterilizing water discharged by the first sterilizing water discharging unit by reflecting the biometric information of the user.

A vitamin D synthesis and sterilization control device according to another embodiment may include a first light source configured to irradiate light for synthesizing vitamin D in the body of a user; a second light source configured to irradiate light for sterilizing a bathroom; a first sterilizing water discharging unit configured to discharge a low-concentration sterilizing water for washing the user; a second sterilizing water discharging unit configured to discharge a high-concentration sterilizing water for cleaning the bathroom; a sensor unit configured to detect whether the user is in the bathroom; and a control unit configured to generate an activation command for controlling at least one of the first light source, the second light source, the first sterilizing water discharging unit and the second sterilizing water discharging unit to be activated based on the detection result on whether the user is in the bathroom.

When the sensor unit detects that the user is in the bathroom, the control unit may generate an activation command for controlling at least one of the first light source and the first sterilizing water discharging unit to be activated.

When the sensor unit detects that the user is not in the bathroom, the control unit may generate an activation command for controlling at least one of the second light source and the second sterilizing water discharging unit to be activated.

The vitamin D synthesis and sterilization control device may further include a communication unit configured to receive an operation command for operating at least one of the first light source and the first sterilizing water discharging unit from a facility in the bathroom, and the control unit may control at least one of the first light source and the first sterilizing water discharging unit to operate based on the operation command.

The vitamin D synthesis and sterilization control device may further include a communication unit configured to receive an operation command for operating at least one of the second light source and the second sterilizing water discharging unit from a portable terminal, and the control unit may control at least one of the second light source and the second sterilizing water discharging unit to operate based on the operation command.

A vitamin D synthesis and sterilization control method of a vitamin D synthesis and sterilization control device according to another embodiment may include: by a sensor unit, detecting whether a user is in a bathroom; and by a control unit, generating an operation command for controlling at least one of a first light source configured to irradiate light for synthesizing vitamin D in the user, a second light source configured to irradiate light for sterilizing the bathroom, a first sterilizing water discharging unit configured to discharge a low-concentration sterilizing water for sterilizing the user and a second sterilizing water discharging unit configured to discharge a high-concentration sterilizing water for sterilizing the bathroom to operate based on the detection result on whether the user is in the bathroom.

A vitamin D synthesis and sterilization control method of a vitamin D synthesis and sterilization control device according to another embodiment may include: by a sensor unit, detecting whether a user is in a bathroom; and by a control unit, generating an activation command for controlling at least one of a first light source configured to irradiate light for synthesizing vitamin D in the user, a second light source configured to irradiate light for sterilizing the bathroom, a first sterilizing water discharging unit configured to discharge a low-concentration sterilizing water for sterilizing the user and a second sterilizing water discharging unit configured to discharge a high-concentration sterilizing water for sterilizing the bathroom to be activated based on the detection result on whether the user is in the bathroom.

According to the present disclosure, a user in the bathroom may enjoy safe and healthy washing by receiving vitamin D as well as using a low-concentration sterilizing water.

In addition, since the bathroom may be cleaned before the user enters the bathroom, when the user enters the bathroom later, the user may enjoy washing in a cleaner environment.

In addition, when the user is not in the bathroom, the bathroom may be cleaned, and when the user is in the bathroom, the user may wash the body and receive vitamin D. Thus, a single device may be selectively used usefully according to the situation.

In addition, since the sterilizing water discharge amount and the irradiation amount of the light source providing vitamin D are adjusted differently according to the biological state of the user, a more suitable washing environment in the bathroom may be provided to the user.

### BRIEF DESCRIPTION OF THE DRAWINGS

FIG. 1 is a block diagram showing a vitamin D synthesis and sterilization control device 1 according to an embodiment.

FIG. 2 is a flowchart for illustrating a vitamin D synthesis and sterilization control method of the vitamin D synthesis and sterilization control device 1 according to an embodiment.

FIG. 3 is a block diagram showing a vitamin D synthesis and sterilization control device 1′ according to an embodiment.

FIG. 4 is a flowchart for illustrating a vitamin D synthesis and sterilization control method of the vitamin D synthesis and sterilization control device 1′ according to an embodiment.

### DETAILED DESCRIPTION

For a following detailed description of the present disclosure, reference is made to the accompanying drawings that show by way of example specific embodiments in which the present disclosure may be practiced. These embodiments are described in sufficient detail to enable those skilled in the art to practice the present disclosure. It should be understood that the various embodiments of the present disclosure are different but need not be mutually exclusive. For example, certain shapes, structures and features described herein may be implemented in other embodiments without departing from the idea and scope of the present disclosure in relation to one embodiment. In addition, it should be understood that locations or arrangements of individual components in each embodiment may be changed without departing from the idea and scope of the present disclosure. Accordingly, the detailed description set forth below is not intended to be taken in a limiting sense, and the scope of the present disclosure, if properly set forth, is limited only by the appended claims, along with any scope equivalent to the claims. Like reference numerals in the drawings refer to the same or similar functions throughout the various aspects.

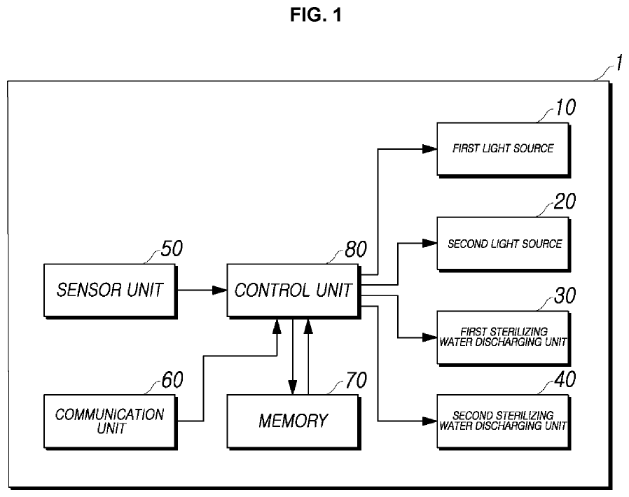

FIG. 1 is a block diagram showing a vitamin D synthesis and sterilization control device 1 according to an embodiment.

As shown in FIG. 1, the vitamin D synthesis and sterilization control device 1 according to an embodiment may include a first light source 10, a second light source 20, a first sterilizing water discharging unit 30, a second sterilizing water discharging unit 40, a sensor unit 50, a communication unit 60, a memory 70, and a control unit 80.

The first light source 10 may irradiate light for synthesizing vitamin D in the body of a user.

The first light source 10 may be configured to irradiate UV-B such as an LED. Preferably, the first light source 10 may be configured to irradiate UV-B having a wavelength of 280 nm to 320 nm. However, light even within other ultraviolet wavelength ranges may also be applied to the present disclosure as long as light for synthesizing vitamin D in the body of the user is irradiated.

One or a plurality of first light sources 10 may be arranged in the vitamin D synthesis and sterilization control device 1.

The second light source 20 may be configured to irradiate light for sterilizing a bathroom, such as an LED. Preferably, the second light source 20 may be configured to irradiate UV-C having a wavelength of 200 nm to 280 nm. However, light even within other ultraviolet wavelength ranges may also be applied to the present disclosure as long as light for maximizing the sterilization effect of the bathroom is irradiated.

One or a plurality of second light sources 20 may be arranged in the vitamin D synthesis and sterilization control device 1.

The first sterilizing water discharging unit 30 may discharge a low-concentration sterilizing water for sterilizing the user.

The first sterilizing water discharging unit 30 may discharge a low-concentration sterilizing water for sterilizing a user who enters the bathroom.

In the present disclosure, the low-concentration sterilizing water is defined as a sterilizing water having an appropriate concentration and/or acidity for washing the body of the user.

Hypochlorite (HOCI) may be used as the low-concentration sterilizing water according to an embodiment, but the present disclosure is not limited thereto.

The second sterilizing water discharging unit 40 may discharge a high-concentration sterilizing water for sterilizing the bathroom.

In the present disclosure, the high-concentration sterilizing water is defined as a sterilizing water having an appropriate concentration and/or acidity for cleaning the bathroom.

Sodium hypochlorite (NaOCl) may be used as the high-concentration sterilizing water according to an embodiment, but the present disclosure is not limited thereto.

The sensor unit 50 may detect whether the user is in the bathroom.

The sensor unit 50 may include a proximity sensor for detecting the body of the user in proximity to the vitamin D synthesis and sterilization control device 1.

In addition, it is also possible to detect whether the user is in the bathroom by using a motion sensor that detects movement or motion of the user.

The communication unit 60 performs data communication with an external device such as a portable terminal (not shown).

The communication unit 60 may support long-distance or short-distance wired/wireless communication between the portable terminal and the vitamin D synthesis and sterilization control device 1. For example, the communication unit 60 may support various communications such as LAN, WAN, WIFI, IrDA, and LTE.

The portable terminal is defined as a device that receives a command from the user and transmits the command to the vitamin D synthesis and sterilization control device 1, such as a smartphone and a tablet.

The memory 70 may store various commands for controlling the operation of the vitamin D synthesis and sterilization control device 1 and/or information obtained as a result of the operation.

The control unit 80 controls the overall operation of each component of the vitamin D synthesis and sterilization control device 1.

The control unit 80 may generate an operation command for controlling at least one of the first light source 10, the second light source 20, the first sterilizing water discharging unit 30 and the second sterilizing water discharging unit 40 to operate based on the detection result on whether the user is in the bathroom.

If the sensor unit 50 detects that the user is in the bathroom, the control unit 80 may generate an operation command for controlling at least one of the first light source 10 and the first sterilizing water discharging unit 30 to operate.

According to an embodiment, the control unit 80 may generate a command for controlling the second light source 20 and the second sterilizing water discharging unit 40 not to operate, together with the operation command.

That is, if the user is in the bathroom, light for synthesizing vitamin D in the body of the user is irradiated through the first light source 10 and/or the low-concentration sterilizing water for washing the body of the user may be discharged.

Meanwhile, since the light for sterilizing the bathroom and the high-concentration sterilizing water for cleaning the bathroom may adversely affect the user, if the user is in the bathroom, the second light source 20 and the second sterilizing water discharging unit 40 are not operated.

As a result, the user in the bathroom may enjoy safe and healthy washing by receiving vitamin D while using the low-concentration sterilizing water.

Meanwhile, if the sensor unit 50 detects that the user is not in the bathroom, the control unit 80 may generate an operation command for controlling at least one of the second light source 20 and the second sterilizing water discharging unit 40 to operate.

According to an embodiment, the control unit 80 may generate a command for controlling the first light source 10 and the first sterilizing water discharging unit 30 not to operate, together with the operation command.

That is, if the user is not in the bathroom, the light for sterilizing the bathroom may be irradiated through the second light source 20 and/or the high-concentration sterilizing water for cleaning the bathroom may be discharged.

Meanwhile, when the user is not in the bathroom, vitamin D synthesis and the low-concentration sterilizing water for washing the user are unnecessary, so the first light source 10 and the first sterilizing water discharging unit 30 are not operated.

As a result, since the bathroom may be cleaned before the user enters the bathroom, when the user enters the bathroom later, the user may enjoy washing in a cleaner environment. In addition, when the user is not in the bathroom, the first light source 10 and the first sterilizing water discharging unit 30 that are unnecessary are not operated, thereby reducing power consumption.

Meanwhile, according to an embodiment, the control unit 80 may generate a command for controlling at least one of the first light source 10, the second light source 20, the first sterilizing water discharging unit 30 and the second sterilizing water discharging unit 40 to operate, if at least one of the first light source 10, the second light source 20, the first sterilizing water discharging unit 30 and the second sterilizing water discharging unit 40 is activated.

That is, the control unit 80 may determine whether at least one of the first light source 10, the second light source 20, the first sterilizing water discharging unit 30 and the second sterilizing water discharging unit 40 is activated first, and then, if it is detected that the user is in the bathroom, the control unit 80 may control the at least one to operate when the at least one is determined as being activated and may control the at least one not to operate when the at least one is determined as being inactivated.

In this case, it is possible to set the at least one not to operate if the at least one is determined as being inactivated, even though it is detected that the user is in the bathroom. As a result, it possible to set an operation limit, which controls not to operate even when it is detected that the user is in the bathroom.

Accordingly, the at least one may not be activated for a user who does not want the corresponding function, and thus, the at least one may not operate even if it is detected that the user is in the bathroom.

Meanwhile, according to the embodiment, the communication unit 60 may receive at least one activation command for activating at least one of the first light source 10, the second light source 20, the first sterilizing water discharging unit 30 and the second sterilizing water discharging unit 40 from the portable terminal, and the control unit 80 may control at least one of the first light source 10, the second light source 20, the first sterilizing water discharging unit 30 and the second sterilizing water discharging unit 40 to be activated based on the activation command received from the portable terminal.

That is, since the activation command may be received from an external device so that activation is determined in advance by the user before entering the bathroom, the user may use a more convenient and more prepared bathroom.

According to another embodiment, the vitamin D synthesis and sterilization control device 1 may include an activation selection switch (not shown) to receive a selection command for the activation selection switch in advance from the user, and the control unit 80 may determine whether or not to activate the vitamin D synthesis and sterilization control device 1 based on the state of the activation selection switch state.

The communication unit 60 may receive biometric information of the user from the portable terminal, and the control unit 80 may reflect the biometric information of the user to control to change at least one of an amount of the light irradiated by the first light source 10 and an amount of the low-concentration sterilizing water discharged by the first sterilizing water discharging unit 30.

Specifically, the control unit 80 may control to change an irradiation intensity and/or an irradiation time of the light of the first light source 10 by reflecting the biometric information of the user, and control to change a discharge time and/or a discharge rate of the low-concentration sterilizing water of the first sterilizing water discharging unit 30.

For reference, here, the portable terminal may include a wearable device capable of measuring a heart rate, a body temperature, biorhythm, and the like of the user.

For example, if it is determined that the user is stable as a result of measuring the biological state of the user, the irradiation amount of the light of the first light source 10 and/or the discharge amount of the low-concentration sterilizing water of the first sterilizing water discharging unit 30 may be relatively increased, whereas if it is determined that the user is unstable, the irradiation amount of the light of the first light source 10 and/or the discharge amount of the low-concentration sterilizing water of the first sterilizing water discharging unit 30 may be relatively decreased.

By enabling different adjustments according to the biological state of the user as above, a more suitable washing environment in the bathroom may be provided to the user.

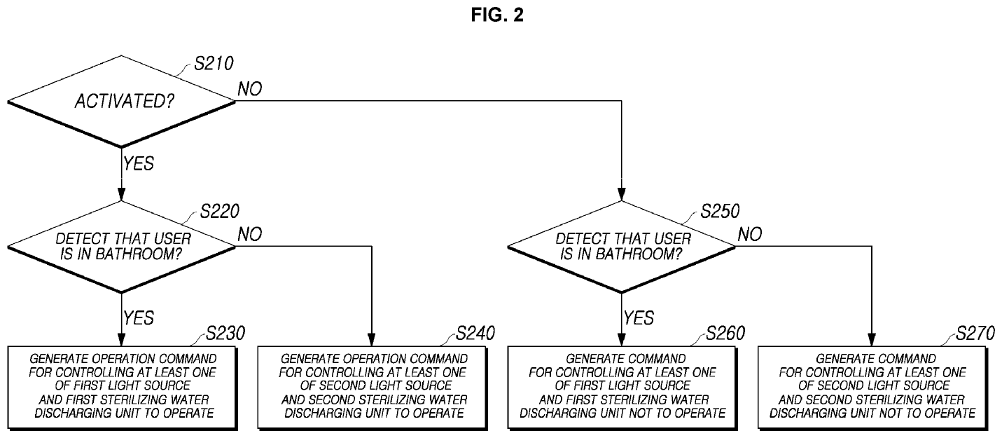

FIG. 2 is a flowchart for illustrating a vitamin D synthesis and sterilization control method of the vitamin D synthesis and sterilization control device 1 according to an embodiment.

As shown in FIG. 2, the control unit 80 may determine whether at least one of the first light source 10, the second light source 20, the first sterilizing water discharging unit 30 and the second sterilizing water discharging unit 40 is activated (S210).

If it is determined that the at least one is activated, it may be determined again whether the user is detected as being in the bathroom (S220), and then different operations may be performed based on the corresponding detection result.

According to an embodiment, the at least one may be activated based on the activation command received from the portable terminal.

When it is determined that the user is the bathroom, the control unit 80 may generate an operation command for controlling at least one of the first light source 10 and the first sterilizing water discharging unit 30 to operate (S230). According to an embodiment, together with the operation command, a command for controlling the second light source 20 and the second sterilizing water discharging unit 40 not to operate may be generated. Accordingly, at least one of the first light source 10 and the first sterilizing water discharging unit 30 may operate, and the second light source 20 and the second sterilizing water discharging unit 40 may not operate.

Meanwhile, if it is determined that the user is not in the bathroom, the control unit 80 may generate an operation command for controlling at least one of the second light source 20 and the second sterilizing water discharging unit 40 to operate (S240). According to an embodiment, together with the operation command, a command for controlling the first light source 10 and the first sterilizing water discharging unit 30 not to operate may be generated. Accordingly, at least one of the second light source 20 and the second sterilizing water discharging unit 40 may operate, and the first light source 10 and the first sterilizing water discharging unit 30 may not operate.

Meanwhile, if it is determined that at least one of the first light source 10, the second light source 20, the first sterilizing water discharging unit 30 and the second sterilizing water discharging unit 40 is not activated, the control unit 80 may determine again whether the user is detected as being in the bathroom (S250), and then different operations may be performed based on the corresponding detection result.

If the user is detected as being in the bathroom, the control unit 80 may generate a command for controlling the first light source 10 and the first sterilizing water discharging unit 30 not to operate (S260). Accordingly, if the at least one is inactivated, even if the user is detected as being in the bathroom, the first light source 10 and the first sterilizing water discharging unit 30 may not operate.

Meanwhile, if it is determined that the user is not in the bathroom, the control unit 80 may generate a command for controlling the second light source 20 and the second sterilizing water discharging unit 40 not to operate (S270). Accordingly, the second light source 20 and the second sterilizing water discharging unit 40 may not operate if the at least one is inactivated and the user is detected as not being in the bathroom.

According to the present disclosure, since it is controlled whether or not to activate each light source 10, 20 and each sterilizing water discharging unit 30, 40 and/or whether or not to operate each light source 10, 20 and each sterilizing water discharging unit 30, 40 according to the detection result of the sensor unit 50 on whether the user is in the bathroom, the present disclosure may be selectively and usefully applied to both users who want and do not want the corresponding function.

Meanwhile, although not shown in FIG. 2, according to another embodiment, the control unit 80 may additionally measure a chlorine concentration in the bathroom even when the vitamin D synthesis and sterilization control device 1 is activated in order to check again whether the bathroom is in a useable state.

To this end, the vitamin D synthesis and sterilization control device 1 according to the present disclosure may additionally include a sensing unit (not shown) for measuring the chlorine concentration and an alarm unit (not shown).

If it is determined that the chlorine concentration in the bathroom measured by the sensing unit (not shown) is less than a predetermined threshold, the steps of FIG. 2 after S220 may be performed. Meanwhile, if it is determined that the chlorine concentration is higher than the predetermined threshold, alarm information for prohibiting the user from entering the bathroom may be provided to the user through the alarm unit (not shown). In addition, the control unit 80 may generate a command for controlling at least one of the second light source 20 and the second sterilizing water discharging unit 40 to operate.

According to this, even when the device is activated, if the chlorine concentration is high, the user cannot enter the bathroom, and thus the bathroom may be used more safely.

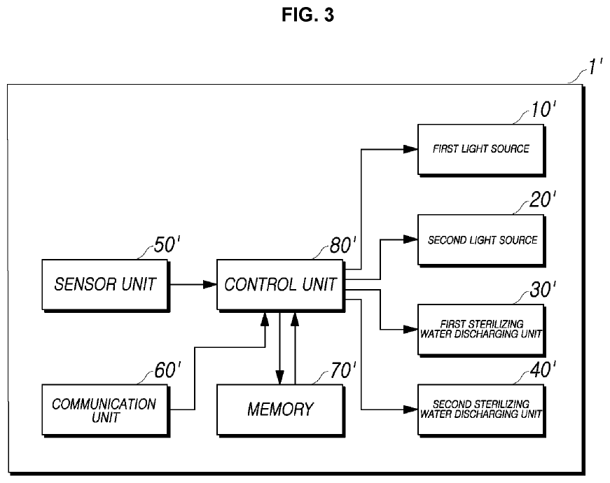

FIG. 3 is a block diagram showing a vitamin D synthesis and sterilization control device 1′ according to an embodiment.

As shown in FIG. 3, the vitamin D synthesis and sterilization control device 1′ according to an embodiment may include a first light source 10′, a second light source 20′, a first sterilizing water discharging unit 30′, a second sterilizing water discharging unit 40′, a sensor unit 50′, a communication unit 60′, a memory 70′, and a control unit 80′.

For each of the components of FIG. 3, the description for each component of FIG. 1 may be applied in the same/similar manner.

The control unit 80′ may generate an activation command for controlling at least one of the first light source 10′, the second light source 20′, the first sterilizing water discharging unit 30′ and the second sterilizing water discharging unit 40′ to be activated based on the detection result on whether the user is in the bathroom.

If the sensor unit 50′ detects that the user is in the bathroom, the control unit 80′ may generate an activation command for controlling at least one of the first light source 10′ and the first sterilizing water discharging unit 30′ to be activated.

According to an embodiment, together with the activation command, the control unit 80′ may generate a command for controlling the second light source 20′ and the second sterilizing water discharging unit 40′ not to be activated.

That is, if the user is in the bathroom, at least one of the first light source 10′ and the first sterilizing water discharging unit 30′ may be activated.

Meanwhile, since the light for sterilizing the bathroom and the high-concentration sterilizing water for cleaning the bathroom may adversely affect the user, the second light source 20′ and the second sterilizing water discharging unit 40′ are not activated if the user is in the bathroom.

On the other hand, the communication unit 60′ may receive an operation command for operating at least one of the first light source 10′ and the first sterilizing water discharging unit 30′ from a facility (not shown) in the bathroom, and the control unit 80′ may control at least one of the first light source 10′ and the first sterilizing water discharging unit 30′ to operate, based on the corresponding operation command.

According to an embodiment, the facility in the bathroom may include a shower, a shower chair, a bathtub, a mirror, or the like.

According to an embodiment, the facility in the bathroom may include a control module (not shown), a detection module (not shown), and a communication module (not shown).

If the facility in the bathroom is a shower, the detection module (not shown) may be provided at a grip portion of the shower.

At this time, the detection module (not shown) may be provided as a sensor applicable to the grip portion of the shower, such as an optical sensor, a button sensor, a proximity sensor.

Meanwhile, if the facility in the bathroom is a chair, the detection module (not shown) may be provided at a buttock seat portion and/or a backrest portion of the shower chair.

In this case, the detection module (not shown) may be provided as a sensor applicable to the shower chair, such as a weight sensor, an optical sensor, a touch sensor, and a proximity sensor.

If contact and/or proximity to the facility in the bathroom by the user is detected by the detection module, the control module may generate an operation command for operating at least one of the first light source 10′ and the first sterilizing water discharging unit 30′ due to the contact and/or proximity and send the operation command to the vitamin D synthesis and sterilization control device 1′.

In addition, the vitamin D synthesis and sterilization control device 1′ may control at least one of the first light source 10′ and the first sterilizing water discharging unit 30′ to operate based on the corresponding operation command.

As a result, since at least one of the first light source 10′ and the first sterilizing water discharging unit 30′ is primarily activated by detecting that the user is in the bathroom and is then secondarily operated based on the operation command for operating at least one of the first light source 10′ and the first sterilizing water discharging unit 30′, the above function is not provided to a user who enters the bathroom but stays in the bathroom for a while without using the facility in the bathroom, thereby reducing power consumption of the vitamin D synthesis and sterilization control device 1′.

Meanwhile, if it is detected that the user is not in the bathroom, the control unit 80′ may generate an activation command for controlling at least one of the second light source 20′ and the second sterilizing water discharging unit 40′ to be activated.

According to an embodiment, together with the activation command, the control unit 80′ may generate a command for controlling the first light source 10′ and the first sterilizing water discharging unit 30′ not to be activated.

In other words, if the user is not in the bathroom, the vitamin D synthesis and the low-concentration sterilizing water for washing the user are unnecessary, so the first light source 10′ and the first sterilizing water discharging unit 30′ are not activated, and only at least one of the second light source 20′ and the second sterilizing water discharging unit 40′ is activated.

Meanwhile, the communication unit 60′ may receive an operation command for operating at least one of the second light source 20′ and the second sterilizing water discharging unit 40′ from the portable terminal, and the control unit 80′ may control at least one of the second light source 20′ and the second sterilizing water discharging unit 40′ to operate, based on the operation command.

As a result, since at least one of the second light source 20′ and the second sterilizing water discharging unit 40′ is primarily activated by detecting that the user is in the bathroom and the above function is secondarily performed based on the operation command for operating at least one of the second light source 20′ and the second sterilizing water discharging unit 40′, the above configuration does not always operate even when the user is not in the bathroom but is set operate only when there is the operation command, thereby reducing power consumption of the vitamin D synthesis and sterilization control device 1′.

According to another embodiment, the vitamin D synthesis and sterilization control device 1′ may include an operation selection switch (not shown) to receive a selection command for the operation selection switch from the user in advance, and the control unit 80′ may determine whether or not to operate the vitamin D synthesis and sterilization control device 1′ based on the state of the operation selection switch.

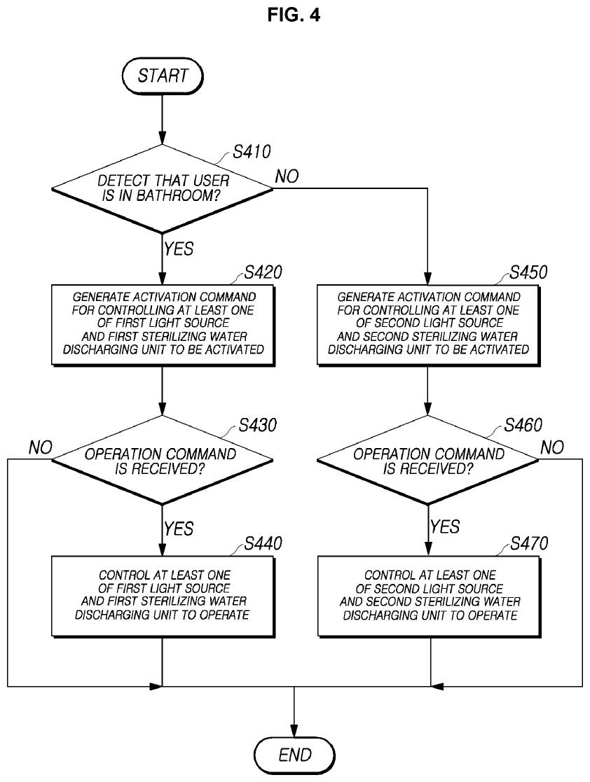

FIG. 4 is a flowchart for illustrating a vitamin D synthesis and sterilization control method of the vitamin D synthesis and sterilization control device 1′ according to an embodiment.

As shown in FIG. 4, the control unit 80′ may generate an activation command for controlling at least one of the first light source 10′, the second light source 20′, the first sterilizing water discharging unit 30′ and the second sterilizing water discharging unit 40′ to be activated or not, based on the detection result on whether the user is in the bathroom (S410).

If the sensor unit 50′ detects that the user is in the bathroom, the control unit 80′ may generate an activation command for controlling at least one of the first light source 10′ and the first sterilizing water discharging unit 30′ to be activated (S420). According to an embodiment, together with the activation command, the control unit 80′ may generate a command for controlling the second light source 20′ and the second sterilizing water discharging unit 40′ not to be activated.

Meanwhile, if the communication unit 60′ receives an operation command for operating at least one of the first light source 10′ and the first sterilizing water discharging unit 30′ from a facility (not shown) in the bathroom, the control unit 80′ may control at least one of the first light source 10′ and the first sterilizing water discharging unit 30′ to operate, based on the corresponding operation command (S430, S440).

On the other hand, if the sensor unit 50′ detects that the user is not in the bathroom, the control unit 80′ may generate an activation command for controlling at least one of the second light source 20′ and the second sterilizing water discharging unit 40′ to be activated (S450).

According to an embodiment, together with the activation command, the control unit 80′ may generate a command for controlling the first light source 10′ and the first sterilizing water discharging unit 30′ not to be activated.

Meanwhile, the communication unit 60′ may receive an operation command for operating at least one of the second light source 20′ and the second sterilizing water discharging unit 40′ from the portable terminal, and the control unit 80′ may control at least one of the second light source 20′ and the second sterilizing water discharging unit 40′ to operate, based on the corresponding operation command (S470).

According to the present disclosure, since it is controlled whether or not to activate based on whether the user is detected as being in the bathroom operate and/or whether or not to operate each light source 10, 20 and each sterilizing water discharging unit 30, 40 based on the operation command in this way, the present disclosure may be selectively applied to the user, and the power consumption of the vitamin D synthesis and sterilization control device 1′ may be reduced.

Meanwhile, although not shown in FIG. 4, according to another embodiment, the control unit 80′ may additionally measure a chlorine concentration in the bathroom before the user enters the bathroom, in order to check again whether the bathroom is in a useable state.

To this end, the vitamin D synthesis and sterilization control device 1′ according to the present disclosure may additionally include a sensing unit (not shown) for measuring the chlorine concentration and an alarm unit (not shown).

If it is determined that the chlorine concentration in the bathroom measured by the sensing unit (not shown) is less than a predetermined threshold, the steps of FIG. 4 after S410 may be performed. Meanwhile, if it is determined that the chlorine concentration is higher than the predetermined threshold, alarm information for prohibiting the user from entering the bathroom may be provided to the user through the alarm unit (not shown).

According to this, even since the user may find whether the chlorine concentration is high before the user enters the bathroom, the user may use the bathroom more safely.

The features, structures, effects, and the like described in the above embodiments are included in one embodiment of the present disclosure, and the present disclosure is not necessarily limited to one embodiment. Furthermore, the features, structures, effects, and the like illustrated in each embodiment may be combined or modified for other embodiments by those skilled in the art. Therefore, the contents related to such combinations and modifications should be interpreted as being included in the scope of the present disclosure.

In addition, although the embodiment has been described above, it is only an example and does not limit the present disclosure, and it will be understood by those skilled in the art that the present disclosure can be changed and modified in various ways beyond the embodiments without deviating from the essential features of the present embodiment. For example, each component specifically illustrated in the embodiment can be modified. In addition, the differences related to these changes and modifications should be construed as being included in the scope of the present disclosure defined in the appended claims.

What is claims is:

1: A vitamin D synthesis and sterilization control device, comprising:
a first light source configured to irradiate light for synthesizing vitamin D in the body of a user;
a second light source configured to irradiate light for sterilizing a bathroom;
a first sterilizing water discharging unit configured to discharge a low-concentration sterilizing water for washing the user;
a second sterilizing water discharging unit configured to discharge a high-concentration sterilizing water for cleaning the bathroom;
a sensor unit configured to detect whether the user is in the bathroom; and
a control unit configured to generate an operation command for controlling at least one of the first light source, the second light source, the first sterilizing water discharging unit and the second sterilizing water discharging unit to operate based on the detection result on whether the user is in the bathroom.

2: The vitamin D synthesis and sterilization control device according to claim 1, wherein when the sensor unit detects that the user is in the bathroom, the control unit generates an operation command for controlling at least one of the first light source and the first sterilizing water discharging unit to operate.

3: The vitamin D synthesis and sterilization control device according to claim 1, wherein when the sensor unit detects that the user is not in the bathroom, the control unit generates an operation command for controlling at least one of the second light source and the second sterilizing water discharging unit to operate.

4: The vitamin D synthesis and sterilization control device according to any one of claim 1, wherein the control unit generates the operation command when at least one of the first light source, the second light source, the first sterilizing water discharging unit and the second sterilizing water discharging unit is activated.

5: The vitamin D synthesis and sterilization control device according to claim 4, further comprising:
a communication unit configured to receive an activation command for activating at least one of the first light source, the second light source, the first sterilizing water discharging unit and the second sterilizing water discharging unit from a portable terminal,
wherein the control unit controls to perform the activation based on the activation command.

6: The vitamin D synthesis and sterilization control device according to claim 2, further comprising:
a communication unit configured to receive biometric information of the user from a portable terminal,
wherein the control unit controls to change at least one of an amount of the light irradiated by the first light source and an amount of the low-concentration sterilizing water discharged by the first sterilizing water discharging unit by reflecting the biometric information of the user.

7: A vitamin D synthesis and sterilization control device, comprising:
a first light source configured to irradiate light for synthesizing vitamin D in the body of a user;
a second light source configured to irradiate light for sterilizing a bathroom;
a first sterilizing water discharging unit configured to discharge a low-concentration sterilizing water for washing the user;
a second sterilizing water discharging unit configured to discharge a high-concentration sterilizing water for cleaning the bathroom;
a sensor unit configured to detect whether the user is in the bathroom; and
a control unit configured to generate an activation command for controlling at least one of the first light source, the second light source, the first sterilizing water discharging unit and the second sterilizing water discharging unit to be activated based on the detection result on whether the user is in the bathroom.

8: The vitamin D synthesis and sterilization control device according to claim 7, wherein when the sensor unit detects that the user is in the bathroom, the control unit generates an activation command for controlling at least one of the first light source and the first sterilizing water discharging unit to be activated.

9: The vitamin D synthesis and sterilization control device according to claim 7, wherein when the sensor unit detects that the user is not in the bathroom, the control unit generates an activation command for controlling at least one of the second light source and the second sterilizing water discharging unit to be activated.

10: The vitamin D synthesis and sterilization control device according to claim 8, further comprising:
a communication unit configured to receive an operation command for operating at least one of the first light source and the first sterilizing water discharging unit from a facility in the bathroom,
wherein the control unit controls at least one of the first light source and the first sterilizing water discharging unit to operate based on the operation command.

11: The vitamin D synthesis and sterilization control device according to claim 9, further comprising:
a communication unit configured to receive an operation command for operating at least one of the second light source and the second sterilizing water discharging unit from a portable terminal,
wherein the control unit controls at least one of the second light source and the second sterilizing water discharging unit to operate based on the operation command.
